(19) United States
(12) Patent Application Publication
(10) Pub. No.: US 2019/0009041 A1
(43) Pub. Date: Jan. 10, 2019

(54) VALVES AND TUBES INCLUDING VALVES

(71) Applicant: SMITHS MEDICAL INTERNATIONAL LIMITED, Ashford (GB)

(72) Inventors: Rabeya Khanom, Bradford (GB); Christopher John Woosnam, Great Sutton (GB); Mansour Youseffi, Bradford (GB)

(73) Assignee: SMITHS MEDICAL INTERNATIONAL LIMITED, Ashford (GB)

(21) Appl. No.: 15/752,447

(22) PCT Filed: Aug. 12, 2016
(86) PCT No.: PCT/GB2016/000150

§ 371 Date: Feb. 13, 2018

(30) Foreign Application Priority Data
Aug. 26, 2015 (GB) 1515123.6

(51) Int. Cl. A61M 16/04; A61M 16/20; F16K 15/06; F16K 15/18; F16K 15/20

(52) U.S. Cl. CPC A61M 16/044; A61M 16/208; A61M 2205/0211; F16K 15/18; F16K 15/20; F16K 15/063

(57) ABSTRACT

An inflation valve (20) for a sealing cuff (10) on a tracheal tube (1) has a helical ceramic spring (40) that urges a valve seal (45, 87) against a valve seat (28, 128). The actuator (51) by which the seal (45, 87) is lifted from the seat to open the valve includes a spring latch mechanism (53). The latch mechanism has three spring fingers (56) that are held resiliently in compression in a recess (60) before the first actuation of the valve. When first used, the actuator is moved forwardly to open the valve so that the free ends of the spring fingers clear the end of the recess and deflect outwardly. When the actuator (51) is released to allow the valve to close, the spring fingers (56) engage a latch surface (63) so that the spring (40) is held in greater tension.

This invention relates to valves of the kind including an outer housing, a valve seat, a valve seal displaceable along the housing into or out of sealing engagement with the valve seat, a resilient member arranged to urge the seal into engagement with the seat and an actuator arrangement extending from the valve seal to an opening of the housing.

Various medico-surgical tubes, such as tracheal tubes, have an inflatable sealing cuff towards the patient end that can be inflated to seal the outside of the tube with a body cavity, such as the trachea. The cuff is inflated and deflated via a small bore lumen extending along the tube that connects towards its machine end with an inflation line terminated by an inflation indicator and a valve. The valve is arranged to remain closed, to block escape of air from the cuff, during use but can be opened by inserting the nose of a syringe or a similar inflation device into the valve to displace a valve element away from a valve seat and allow air to be supplied to or from the valve to inflate or deflate the sealing cuff. It is important that the valve work reliably so that air does not leak out from the cuff and so that it can be opened readily to allow the cuff to be deflated, such as when the tube needs to be removed. Usually, these valves include a spring element in the form of a helical metal wire to urge the valve element to the seated, closed position. One problem with valves including such a spring element is that they may not be compatible with MRI scanners. Although it is possible to use a non-metallic spring element, such as of a resilient plastics material, these can be difficult to manufacture reliably. In particular, thermoplastics can be susceptible to shrinkage after removal from the mould or as a result of sterilisation or ageing after storage. One form of valve is described in PCT/GB2016/000011.

Valves are also used in other applications in addition to sealing cuffs of medico-surgical tubes.

It is an object of the present invention to provide an alternative valve and a medico-surgical tube including such a valve.

According to the present invention there is provided a valve of the above-specified kind, characterised in that the actuator arrangement includes a latch member and is displaceable forwardly from an initial position before use where it is out of engagement with a latch surface on the housing to a second position where the latch member engages the latch surface and prevents movement of the actuator arrangement rearwardly beyond the latched position, that the actuator arrangement is displaceable forwardly from the latched position to displace the seal away from the seat and allow flow through the housing, and that the actuator arrangement, when released, allows the seal to be displaced by the action of the resilient member to return to engagement with the seat and prevent flow along the valve.

The latch member may include an outwardly displaceable spring element that is held inwardly in the initial position by engagement with an external surface formation and that moves resiliently outwardly when displaced forwardly to clear the surface formation, thereby enabling the spring element to engage the latch surface. The resilient member is preferably a non-metallic member, such as a ceramic and may be a helical spring. The valve may includes a cylindrical support member secured in the forward end of the outer housing, the support member having a rear projecting nose and an axial bore and the forward end of the helical spring embracing the rear projecting nose. Alternatively, the resilient member may be a cylindrical member of a plastics material.

According to another aspect of the present invention there is provided a medico-surgical tube including an inflatable sealing cuff encircling the tube, an inflation lumen extending along the tube and opening into the cuff at one end and a valve connected at the opposite end of the inflation lumen to allow or prevent gas flow to or from the cuff, the valve being a valve according to the above one aspect of the present invention.

The tube may be a tracheal tube.

A tracheal tube including a valve according to the present invention will now be described, by way of example, with reference to the accompanying drawings, in which:

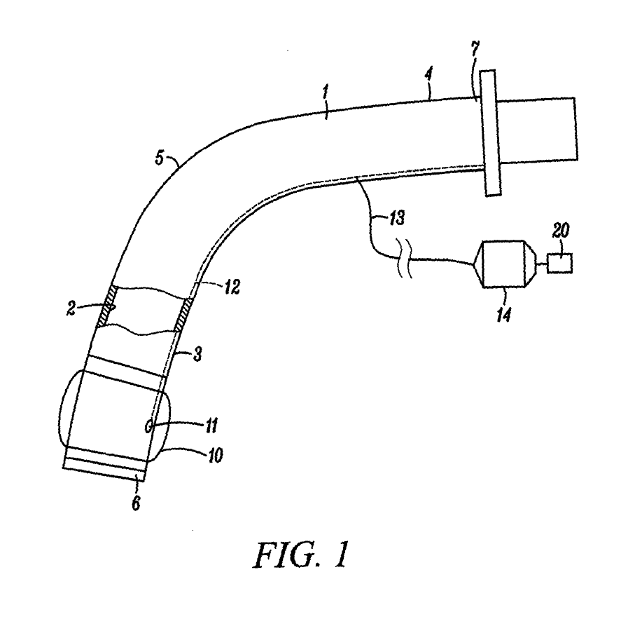

FIG. 1 is a side elevation view of a tracheostomy tube;

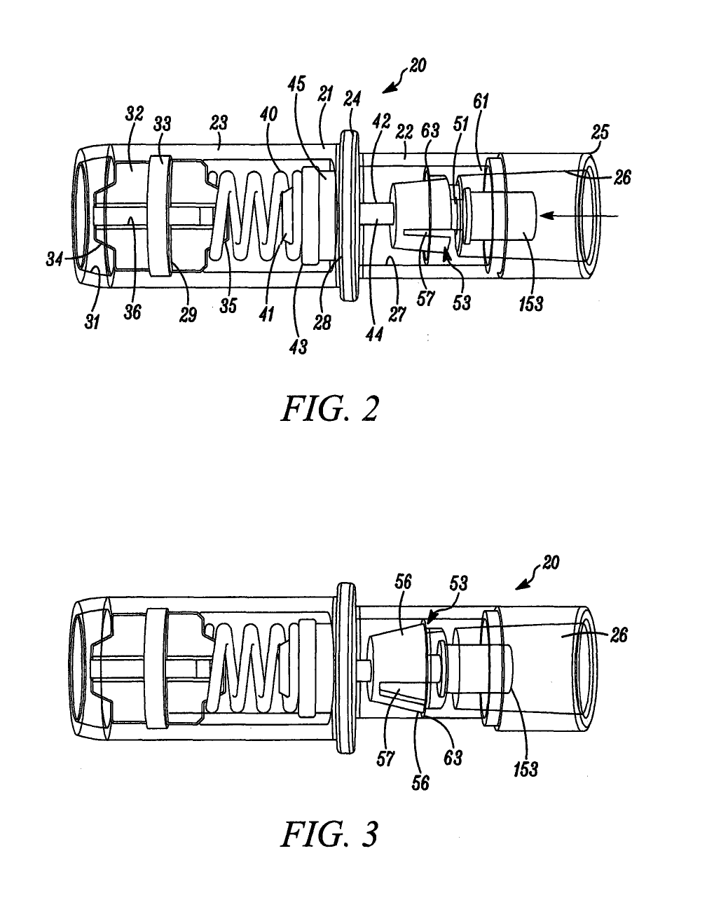

FIG. 2 is a cutaway side elevation view of a first form of valve in an initial condition before actuation;

FIG. 3 is a cutaway side elevation an end view of the valve of FIG. 2 when activated but closed;

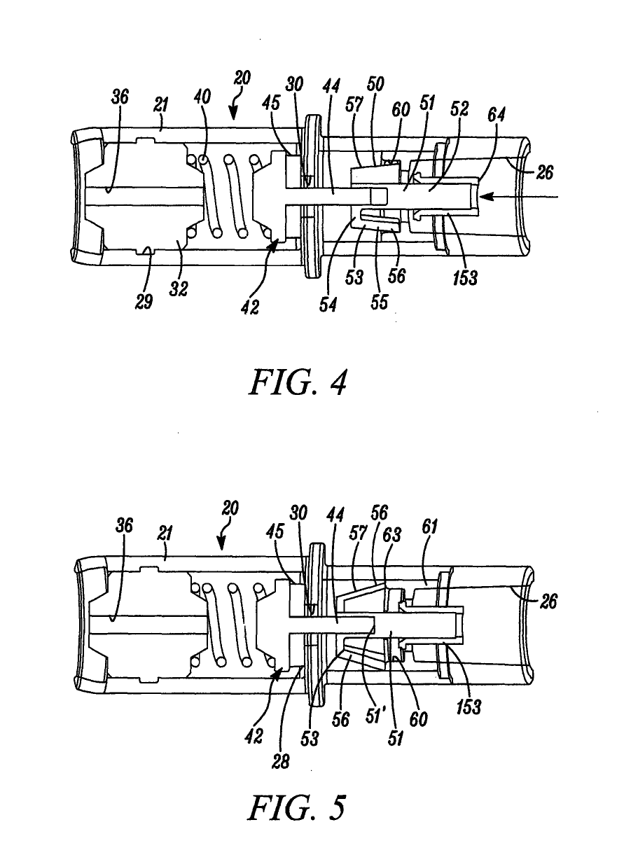

FIG. 4 is a cross-sectional side elevation view of the valve in the initial condition shown in FIG. 2;

FIG. 5 is a cross-sectional side elevation view of the valve in the activated condition shown in FIG. 3;

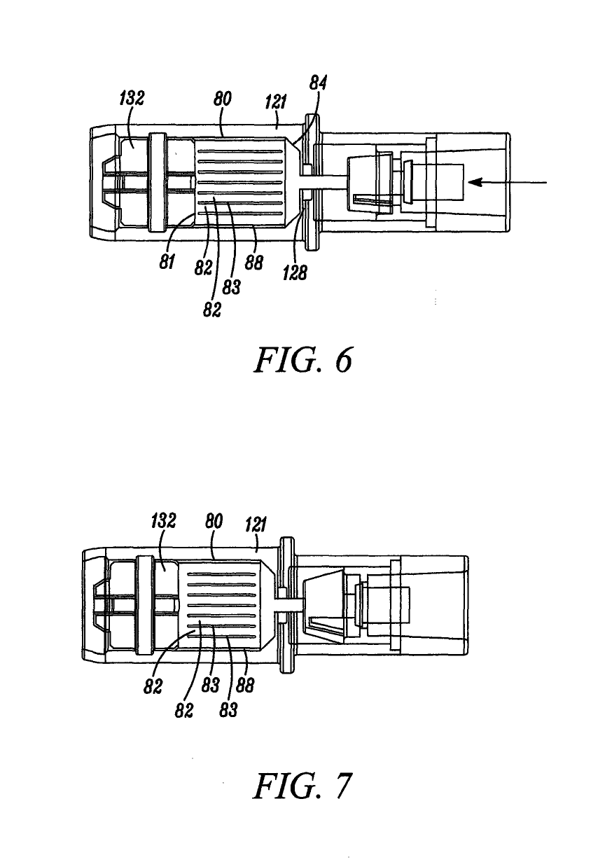

FIG. 6 is a cutaway side elevation view of a second form of valve in an initial condition;

FIG. 7 is a cutaway side elevation view of the second form of valve in the activated condition;

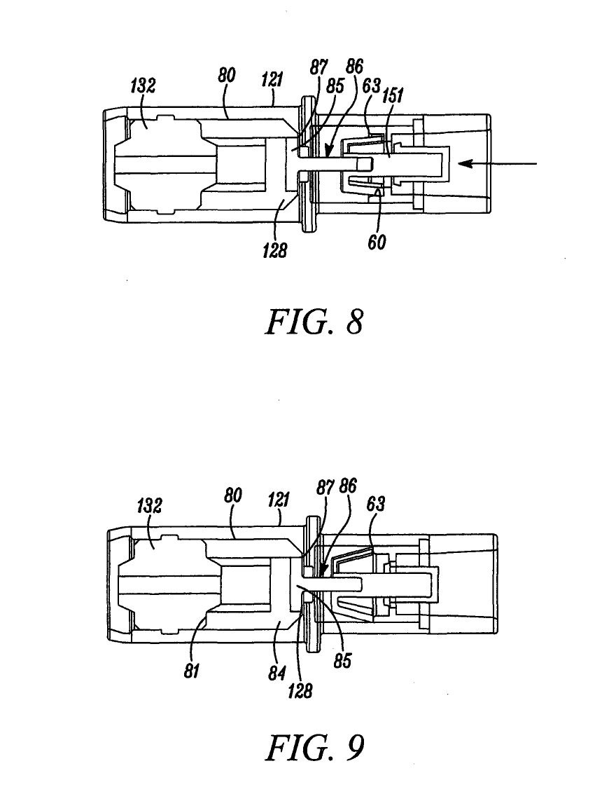

FIG. 8 is a cross-sectional side elevation view of the second form of valve in the initial condition shown in FIG. 6; and

FIG. 9 is a cross-sectional side elevation view of the second form of valve in the activated condition shown in FIG. 7.

With reference first to FIG. 1, the tracheostomy tube includes a tubular shaft 1 having a bore 2 extending along its length. The tube is formed with a relatively straight patient end portion 3 and a relatively straight machine end portion 4 linked by a curved intermediate portion 5 so that the patient and machine ends 6 and 7 are angled at about 100° to one another. The shaft 1 is extruded or moulded from a plastics material such as PVC or silicone. Towards its patient end 6 the tube has sealing means provided by an inflatable cuff 10 embracing the shaft 1. The cuff 10 is of the high-volume/low-pressure kind so that it has a relatively floppy shape when deflated but, when inflated, it fills out at low pressure to a diameter just larger than the internal diameter of the trachea, so that it contacts the inside of the trachea with low pressure. The cuff 10 is moulded to the desired inflated shape from a plastics material such as PVC or polyurethane. The cuff 10 is attached to the shaft 1 at opposite ends over an opening 11 on the outer surface of the shaft into an inflation lumen 12 extending along the shaft within its wall thickness. The inflation lumen 12 is connected towards the rear end 7 of the tube with a small-bore inflation line 13 that is terminated by an inflation indicator 14 and a valve 20.

With reference now also to FIGS. 2 to 5, the valve 20 is of a generally cylindrical shape and circular section having an outer housing 21 of tubular shape with a rear portion 22, a forward portion 23 and a short, radially-projecting annular flange 24 between them. The forward and rear portions 23 and 22 have a constant external diameter with the forward portion being slightly larger in diameter than the rear portion. The rear end 25 of the housing 21 opens into a luer-tapered bore 26, which extends along just over half the length of the rear portion 22 and continues as a section 27 of constant diameter to an internal ledge 28 located level with the external flange 24. The ledge 28 defines a circular opening 30 (most clearly seen in FIGS. 4 and 5) about half the diameter of the opening at the rear end 25. The bore through the forward part 23 of the housing 21, forwardly of the ledge 28, has a constant circular diameter apart from an annular groove 29 close to its forward end and a slightly enlarged and tapered recess 31 at its open forward end. The forward part 23 of the housing 21 contains a generally cylindrical support member 32 secured fixedly in the housing 21.

The support member 32 has an annular, projecting rib 33 midway along its length that engages and locates in the groove 29 to prevent movement along the housing 21. The support member 32 is typically bonded in the housing 21 by means of an adhesive, solvent or the like. At opposite ends, the support member 32 is formed with respective forward and rear projecting nose formations 34 and 35. A gas passage in the form of an axial bore 36 extends along the support member 32 and opens through the respective nose formations 34 and 35. The rearwardly-facing, right-hand nose formation 35 acts as a location to retain the forward end of a helical spring 40 that extends around the nose formation. The spring 40 has a circular section and extends along a helical path of circular section that extends axially of the housing 21. The spring 40 is made of an MRI-safe material, such as a non-magnetic and non-metallic material. Preferably, the spring 40 is of a ceramic material such as silicon nitride or a ceramic mixture including zirconia. Alternatively, the spring could be of a resilient polymer. The opposite, rear end of the spring 40 embraces a forwardly-facing nose formation 41 of the same shape as the nose formation 35 and formed at the forward end of an axially-displaceable valve member 42. The nose formation 41 projects centrally from a radial supporting disc 43 at the forward end of a valve stem 44. The valve member 42 supports a valve seal 45 in the form of an annular disc of a resilient, compliant material such as a soft, rubber-like material. The valve seal 45 embraces the valve stem 44 and its forward face lies against the rear face of the supporting disc 43. The opposite, rearwardly-facing surface of the valve seal 45 lies in contact with the valve seat provided by the forward surface of the ledge 28 around the opening 30 when the valve is in a closed state. The valve member 42 can be displaced forwardly against the resilience of the spring 40 to lift the seal 45 out of contact with the valve seat 28 and open the valve 20 in a manner described in more detail below.

The rear end of the stem 44 of the valve member 42 is slidably received in an axial cavity 50 (FIGS. 4 and 5) extending in an actuator member 51 along about one third of its length and having a floor 51′. The actuator member 51 is moulded from a stiff, resilient plastics material such as polyester. The actuator member 51 comprises a central, axial shaft 52 of circular section into which the cavity 50 extends. At its forward, left-hand end the shaft 52 is formed with a latch member 53 of a generally cylindrical, can shape having a radially-extending floor 54 at one end and a circular wall 55 extending rearwardly coaxially of the shaft 52. The wall 55 is divided into three spring elements or fingers 56 by three equally spaced longitudinal slots 57. In its natural condition, when unconstrained, the wall 55 flares outwardly at an angle of about 30° so that it has a larger diameter at its rear end than at its forward end. In the position shown in FIGS. 2 and 4, however, the valve 20 is in its initial, inactivated state and the latch member 53 is constrained by being in a rear position where the rear end of the spring fingers 56 extend within a reduced diameter circular recess 60 formed in the forward end of a cylindrical insert 61 bonded into the rear end of the section 27 of the bore through the rear portion 22 of the housing 21. The forward end of the insert 61 provides an annular latch surface 63. The rear end of the spring fingers 56 locate against an internal ledge 62 within the insert 61. The diameter of the recess 60 is less than the natural, unconstrained diameter of the rear end of the wall 55 on the latch member so that the spring fingers 56 are compressed inwardly. This acts to retain the latch member 53 by friction in the rear position until activated by the user. The rear end of the shaft 52 of the latch member 53 has a cap 153 bonded to it where it projects along the luer tapered bore 26. The rear end 64 of the cap 153 is cut away or otherwise profiled to provide a non-occluding surface to ensure free passage of fluid around the rear end of the cap when it is engaged by the nose of a syringe or the like.

The valve 20 is supplied to the user and is stored before use in the initial, inactivated position shown in FIGS. 2 and 4. In this position it can be seen that the rear end of the stem 44 of the valve member 42 is spaced from the floor 51′ of the cavity 50 of the actuator member 51 so that no force is applied by the actuator member to the valve member. The dimensions of the spring 40 and the position of the support member 32 and the forward end of the valve member 42 are such that the spring is just sufficiently compressed to ensure that the seal 45 is sealingly engaged with the valve seat 28. This ensures that the spring 40 and seal 45 are not degraded by being highly loaded for long periods during storage.

When the tracheostomy tube 1 is inserted in the patient and the cuff 10 needs to be inflated for the first time the user inserts the nose of a syringe into the luer bore 26 of the valve 20. The nose of the syringe displaces the actuator member 51 and the latch member 53 but initially does not displace the valve member 42 because of the separation between the rear end of the valve member stem 44 and the floor 51′ of the cavity 50. The latch member 53 moves forwardly until the rear end of the latching spring fingers 56 clear the forward end of the recess 60, thereby allowing the rear end of the spring fingers to snap outwardly to engage the latching surface 63 in the position shown in FIGS. 3 and 5. In this position, the actuator member 51 cannot be pulled rearwardly again because the rear end of the spring fingers 56 of the latch member 53 engages the latch surface 63 so, if the syringe were to be removed, the valve 20 would remain in the activated but still closed state shown in FIGS. 3 and 5. In this activated state the rear end of the stem 44 of the valve member 42 is engaged by the floor 51′ of the cavity 50 causing the valve member to be displaced a small distance. This is not enough to open the valve 20 since the seal 45 is relaxed slightly but insufficiently to allow flow between it and the valve seat 28. The spring 40 is put under slightly more tension than in the inactivated state.

To inflate the cuff 10 the syringe is pushed further into the valve 20 to a position where the outside of the nose of the syringe makes a luer slip fit with the bore 26. This displaces the actuator member 51 and valve member 42 together, thereby lifting the seal 45 off the valve seat 28 to open the valve and allow fluid, such as air or water to flow around the actuator member when the plunger of the syringe is depressed. Fluid flows through the opening 30, around the seal 45, through the turns of the spring 40 and along the bore 36 through the support member 32 and into the inflation indicator 14 attached to the forward end of the valve 20. From there, the fluid flows along the inflation line 13 and inflation lumen 12, out of the opening 11 and into the interior of the cuff 10. Fluid can also flow in the opposite direction.

Instead of using a helical spring to provide a resilient force urging the valve seal against a seat it would be possible to use other resilient means. FIGS. 6 to 9 show an alternative valve having an identical form of actuator and latch as in the embodiment described above. Components identical to those in FIGS. 2 to 5 are given the same numerals but with the addition of 100. In place of the helical spring used in the first embodiment, the resilient means is provided by a resilient cylinder 80 of a plastics material. The cylinder 80 is hollow and its forward end 81 is open and rests around the outside of a support member 132 fixed at the forward end of the housing 121. The outer surface of the cylinder 80 is formed with a pattern of longitudinal grooves 82 and ribs 83 to ensure that there is always a fluid passage between the outside of the cylinder and the inside of the housing 121. The cylinder 80 is also formed with several slots 88 extending completely through the thickness of the wall of the cylinder to allow flow between the inside and outside of the cylinder. The rear end 84 of the cylinder 80 is bonded to an enlarged circular head 85 (FIGS. 8 and 9) at the forward end of a valve member 86 similar to the valve member 42 in the first embodiment. The rear end 84 of the cylinder 80 extends radially around the outside of the head 85 to provide a narrow, rearwardly-facing resilient ring 87 that provides the valve seal and that engages in a sealing manner with the forward surface of the valve seat 128.

When the actuator member 151 is pushed forwardly, such as by the nose of a syringe, to open the valve, the valve member 86 is pushed forwardly to move the valve seal 87 forwardly away from the valve seat 128. This causes the resilient cylinder 80 to buckle slightly inwardly as it is compressed axially. Fluid can flow between the valve seal 87 and the seat 128 and between the inside of the housing 121 and the outside of the cylinder along the grooves 82 and through the slots to the inside of the cylinder so that it can then flow along the bore 134 through the support member.

The arrangement of the present invention enables a non-metallic, MRI-compatible resilient element to be used although the resilient element in the valve could be a conventional helical metal wire spring. The present invention provides a valve that can be latched into an operational position after an initial actuation. This arrangement allows for compensation of any dimensional change, especially shrinkage, of the plastic components of the valve that might take place over time such as after sterilisation or caused by ageing after prolonged storage. The valve components can be readily manufactured using conventional techniques, such as injection blow moulding. The invention accommodates variations in tolerances between components without affecting the performance of the valve, thereby enabling manufacturing costs to be kept to a minimum.

1-9. (canceled)

10. A valve including an outer housing, a valve seat, a valve seal displaceable along the housing into or out of sealing engagement with the valve seat, a resilient member arranged to urge the seal into engagement with the seat and an actuator arrangement extending from the valve seal to an opening of the housing, characterised in that the actuator arrangement includes a latch member and is displaceable forwardly from an initial position before use where it is out of engagement with a latch surface on the housing to a second position where the latch member engages the latch surface and prevents movement of the actuator arrangement rearwardly beyond the latched position, that the actuator arrangement is displaceable forwardly from the latched position to displace the seal away from the seat and allow flow through the housing, and that the actuator arrangement, when released, allows the seal to be displaced by the action of the resilient member to return to engagement with the seat and prevent flow along the valve.

11. A valve according to claim 10, characterised in that the latch member includes an outwardly displaceable spring element that is held inwardly in the initial position by engagement with an external surface formation and that moves resiliently outwardly when displaced forwardly to clear the surface formation, thereby enabling the spring element to engage the latch surface.

12. A valve according to claim 10, characterised in that the resilient member is a non-metallic member.

13. A valve according to claim 12, characterised in that the resilient member is of a ceramic.

14. A valve according to claim 10, characterised in that the resilient member is a helical spring.

15. A valve according to claim 14, characterised in that the valve includes a cylindrical support member secured in the forward end of the outer housing, that the support member has a rear projecting nose and an axial bore, and that the forward end of the helical spring embraces the rear projecting nose.

16. A valve according to claim 10, characterised in that the resilient member is a cylindrical member of a plastics material.

17. A medico-surgical tube including an inflatable sealing cuff encircling the tube, an inflation lumen extending along the tube and opening into the cuff at one end and a valve connected at the opposite end of the inflation lumen to allow or prevent gas flow to or from the cuff, characterised in that the valve comprises an outer housing, a valve seat, a valve seal displaceable along the housing into or out of sealing engagement with the valve seat, a resilient member arranged to urge the seal into engagement with the seat and an actuator arrangement extending from the valve seal to an opening of the housing, wherein the actuator arrangement includes a latch member and is displaceable forwardly from an initial position before use where it is out of engagement with a latch surface on the housing to a second position where the latch member engages the latch surface and prevents movement of the actuator arrangement rearwardly beyond the latched position, that the actuator arrangement is displaceable forwardly from the latched position to displace the seal away from the seat and allow flow through the housing, and that the actuator arrangement, when released, allows the seal to be displaced by the action of the resilient member to return to engagement with the seat and prevent flow along the valve.

18. A tube according to claim 17, characterised in that the tube is a tracheal tube.

19. A medico-surgical tube according to claim 17, characterised in that the latch member includes an outwardly displaceable spring element that is held inwardly in the initial position by engagement with an external surface formation and that moves resiliently outwardly when displaced forwardly to clear the surface formation, thereby enabling the spring element to engage the latch surface.
